(19) United States
(12) Patent Application Publication
(10) Pub. No.: US 2009/0306537 A1
(43) Pub. Date: Dec. 10, 2009

(54) System and Method For Detecting The Phase Of A Female Menstrual Cycle

(76) Inventor: Andy Ofer Goren, Newport Beach, CA (US)

Correspondence Address: Nixon Peabody LLP, P.O. Box 60610, Palo Alto, CA, 94306, US

(21) Appl. No.: 12/481,520

(22) Filed: Jun. 9, 2009

Related U.S. Application Data
(60) Provisional application No. 61/060,085, filed on Jun. 9, 2008.

(51) Int. Cl. A61B 10/00

(52) U.S. Cl. 600/551

(57) ABSTRACT

A system for predicting a woman's current position in her menstrual or ovulation cycle includes a baseline voice characteristics storage module in which first set of characteristics of the woman's voice at a known time relative to her menstrual or ovulation cycle is stored, an analysis module for receiving for analyzing a voice sample from the woman and generating a second set of characteristics of the woman's voice, a comparison module for comparing the first and second sets of characteristics, and a results module for providing a prediction of the woman's current position in her menstrual or ovulation cycle based on the comparison.

### CROSS-REFERENCE TO RELATED APPLICATIONS

This application claims the benefit of provisional patent application No. 61/060,085 filed Jun. 9, 2008, entitled “System and Method For Detecting The Phase Of A Female Menstrual Cycle.”

### BACKGROUND OF THE INVENTION

1. Field of the Invention

The invention relates to a system and method for detecting the phase of a female menstrual cycle.

2. Description of the Related Art

Accurately and easily predicting and detecting the peak time of ovulation in a human female is important for sexual reproduction as well as avoidance of fertilization when having unprotected sexual intercourse.

Current trends in human reproduction point to increased difficulty in fertilization for unknown reasons; hence, various tools have been invented to assist female subjects to accurately predict the optimal time for fertilization/ovulation. It is also advantageous for a woman to learn when unprotected sex is less likely to result in fertilization.

In the United States over $9.5 billion dollars are spent annually on infertility products and treatments. Many fertility and ovulation products are either not used properly or timely due to cumbersome collection procedures (such as urine or cervical mucus examination) and are not practical for a busy and/or working woman. Some ovulation detection methods such as calendars are inaccurate because ovulation does not always occur at the same interval period every month and yet other methods such as temperature detection are inaccurate due to an individuals varying temperature with external and internal conditions unrelated to fertility.

The aim of the current invention is to provide women with a simple, easy to use, and accurate diagnostic of a woman's current menstrual cycle phase and her chance for fertilization.

### DETAILED DESCRIPTION

The description herein is provided in the context of a diagnostic for human female ovulation. Those of ordinary skill in the art will realize that the following detailed description is illustrative only and is not intended to be in any way limiting. Other embodiments will readily suggest themselves to such skilled persons having the benefit of this disclosure. Reference will now be made in detail to implementations as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

The female's larynx contains several types of hormonal receptors. A woman's voice is shaped by the affect of progesterone and estrogen on the larynx during puberty.

Various scientific experiments provide evidence that a woman's voice changes during the menstrual cycle in response to changing hormonal concentrations. These changes can be observed during different phases of the menstrual cycle as well as menopause. In fact, hormonal contraceptives reduce the minute changes in a woman's voice induced by varying hormonal levels during the menstrual cycle.

In accordance with one approach described herein, a woman creates a personal account through a world wide web portal. At the beginning of the menstrual cycle a woman calls a personal number (via telephone, mobile phone, or Internet phone) and enters her account number. The woman speaks to the system a specified sentence for a specified time duration. The system records the woman's voice and analyzes it using a Fourier Transform of the frequencies of the woman's voice, or performs a different sound and/or speech processing procedure. The system then stores the voice recording and accompanying analysis. The system may subsequently call the woman at a specified time or every day or randomly and request to obtain a subsequent voice sample, preferably of the same sentence. Alternatively, the woman can call the system at any time to provide the voice sample. The system records and stores the subsequent voice sample(s) and any analyses thereof that are also conducted. The system then compares the voice subsequent sample(s) and/or analyses with the initial sample and/or analysis and generates a probability that the woman is ovulating. It may be that a higher probability of ovulation is generated if the pitch of the woman's voice has increased, or it is higher than the average pitch of her woman voice recorded over several calls.

In yet another embodiment, a database of voices, voice frequency analysis, and a women's ovulation cycle is generated from sampling a plurality of women. The database also contains, for each woman, differences in voice frequencies at different times relative to reference date associated with her ovulation cycle, such as her first menstruation date or her first ovulation date. When a woman calls the system and speaks a specified sentence, the system analyzes her voice to compare voice frequencies to those of other women's base frequency, and generates a profile based on a reference date. When the woman calls the system at another date and records her voice, system analyzes and compares the difference between voice the frequencies of voice at the two calls, and uses the difference to compare to the reference database. Based on similarity of voice differences to voice frequency differences in the reference database, the system generates a probability of a woman's ovulation, or alternatively a risk for getting pregnant or not getting pregnant. It is understood that the database can be generated in many ways, one of which is collecting ovulation data using existing diagnostics devices and voice recordings and creating voice frequency differences based on such data and voice recordings.

In yet another embodiment a woman does not call the system, but the system uses the woman's voice from an Internet audio file, a Internet chat, or a any voice medium such as a television or radio transmission.

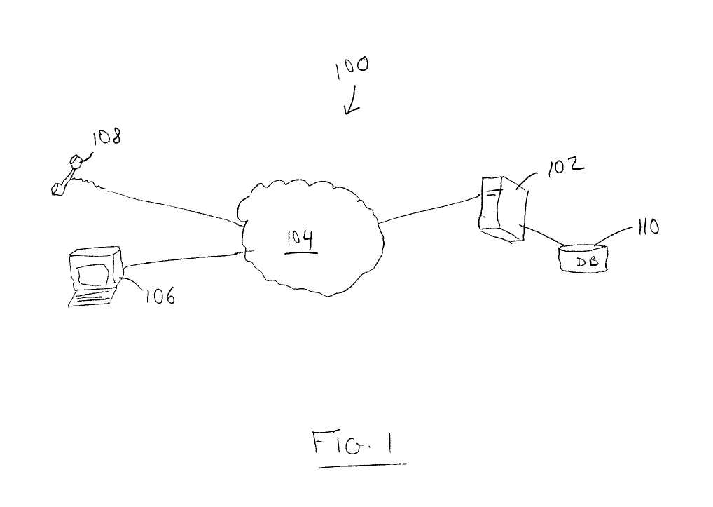

FIG. 1 illustrates a system 100 in which a woman interacts with a service that is hosted by a server 102. The service operates to a provide the woman (or a caregiver) with indication of her location on the menstrual cycle and/or relative to ovulation. The interaction takes place through a network 104, such as the Internet or a cellular or satellite network, and is interfaced, at the woman's end, through a user device 106 such as a personal computer or laptop or the like, and/or through a telephone 108, which may be cellular telephone or a landline. A voice-over-IP application is contemplated when the user device is a desktop or laptop computer or the like. The server 102 has access to a database 1 10, containing for example a previously recorded “baseline” version of the woman's voice, or that other women, in the manner described above.

It may also be possible to use a neural network to implement the system and method, to predict the likelihood that the woman is ovulating based on a woman voice profile. According to such an approach, for predicting likelihood of a woman's likely to ovulate or get pregnant can include (a) constructing an N-layer neural network, and (b) training the neural network with a data set of women voices at different times during their menstrual cycle, (c) obtaining a voice sample from the subject (d) generating a voice-based profile from the sample, the profile being a function of values associated with a prescribed set of voice frequencies; (e) obtaining a difference vector from the profile compared to a reference time during the menstrual cycle; (f) inputting the difference vector into the neural network.

The above are exemplary modes of carrying out the invention and are not intended to be limiting. It will be apparent to those of ordinary skill in the art that modifications thereto can be made without departure from the spirit and scope of the invention as set forth in the following claims.

What is claimed:

1. A system for predicting a woman's current position in her menstrual or ovulation cycle, comprising:
a baseline voice characteristics storage module in which first set of characteristics of the woman's voice at a known time relative to her menstrual or ovulation cycle is stored;
an analysis module for receiving for analyzing a voice sample from the woman and generating a second set of characteristics of the woman's voice;
a comparison module for comparing the first and second sets of characteristics; and
a results module for providing a prediction of the woman's current position in her menstrual or ovulation cycle based on the comparison.

2. The system of claim 1, wherein the first and second sets relate frequency and or pitch.

3. The system of claim 1, wherein the comparison module further compares the difference between the first and second sets of characteristics with pre-stored population differences.

4. The system of claim 1, wherein a neural network is employed.
